(19) United States
(12) Patent Application Publication
(10) Pub. No.: US 2009/0082642 A1
(43) Pub. Date: Mar. 26, 2009

(54) SYSTEM AND METHOD FOR MEASUREMENT OF BIOLOGICAL PARAMETERS OF A SUBJECT

(75) Inventor: Ilya FINE, Rehovot (IL)

Correspondence Address: HOUSTON ELISEEVA, 4 MILITIA DRIVE, SUITE 4, LEXINGTON, MA, 02421, US

(73) Assignee: ELFI-TECH LTD., Rehovot (IL)

(21) Appl. No.: 12/330,098

(22) Filed: Dec. 8, 2008

Related U.S. Application Data
(63) Continuation of application No. PCT/IL2007/000710, filed on Jun. 13, 2007.
(60) Provisional application No. 60/812,973, filed on Jun. 13, 2006.

(51) Int. Cl. A61B 5/00

(52) U.S. Cl. 600/300

(57) ABSTRACT

Disclosed is a system and method for use in monitoring of biological parameters of a subject. The system includes an illumination unit including at least one light source of at least one pre-selected wavelength band, to be applied to a selected region in the subject; and a detection system configured for measuring reflections of the light at different angles and different spatial locations with respect to the illuminated region. The detection system is configured and operable to detect spatially separated light components corresponding to the specular dependent component of the signal and the pulsatile-related diffused component of the signal coming from the subject in different directions respectively, thereby defining at least two independent channels of information, enabling identification of the reflected signal part dependent on motion effects.

### RELATED APPLICATIONS

This application is a Continuation of PCT application serial number PCT/IL2007/000710, filed on Jun. 13, 2007, which in turn claims the benefit under 35 USC 119(e) of U.S. Provisional Application No. 60/812,973, filed on Jun. 13, 2006, both of which are incorporated herein by reference in their entirety.

### FIELD OF THE INVENTION

The present invention is generally in the field of optical measurement techniques on a subject used in medical and other applications, and relates to a system and method capable of distant or non-contact monitoring of the biological parameters of a subject.

### BACKGROUND OF THE INVENTION

A photoplethysmograph, i.e. an optical volumetric measurement of an organ, is often obtained by using a pulse oximeter which illuminates the skin and measures changes in light absorption. A conventional pulse oximeter monitors the perfusion of blood to the dermis and subcutaneous tissue of the skin. The change in volume is detected by illuminating the skin and then measuring the amount of light either transmitted or reflected to a photodiode. Each cardiac cycle appears as a peak. The shape of the photoplethysmograph waveform differs from subject to subject, and varies with the location and manner in which the pulse oximeter is attached. Motion artifact corruption of near infrared plethysmography, causing both measurement inaccuracies and false alarm conditions, is a primary restriction in the current clinical practice and future applications of this useful technique. The most disturbing motion artifact results from a frequently occurring unpredictable relative mechanical movement between an optical sensor and the subject.

Therefore it is a common practice in non-invasive optical measurement techniques that a sensor is physically attached and coupled to the human body under measurements. A typical sensor of this kind (pulse-oximeter) consists of light sources (LEDs, for example) emitting light in the area of visible and near infrared spectrum, and a light detector or plurality of light detectors (in general, detection module). All these elements are an integral part of a one complete enclosure. Only when correctly attached to a subject, such pulse-oximeter system is considered to be in a proper condition to proceed with the measurements. Once the system is fixed, the measurement starts. An optical response of the body is detected, and pulsatile or another biological related signal component is extracted and used to provide information addressing a heart rate, level of blood perfusion, arterial blood oxygen saturation, blood pressure and other physiological parameters.

There are two different types of configuration of a non-invasive optical measurement system. The first type measurement set-up operates with the so-called transmission mode, where a perfused tissue is positioned between a light source unit (2 LEDs matrix for instant) and a detection module. This configuration is achieved by using a finger clip for example. Other popular body locations for transmission-mode measurements include an ear lobe for adults and toes for neonatal monitoring. The second type measurement set-up operates with reflection mode, and can be used, in principle, at any location of the body. For example, forehead or chest location is considered as a popular one.

Either for transmission mode or for reflection set-up, the problem of motion artifacts is reduced by securing a tight contact between the sensor and the body skin. In the case where the optical and mechanical coupling between the sensor and body surface is weak, a very strong motion artifact may drastically reduce the quality of the measured signal.

It is clear that motion artifact is an inherent problem for any distant or non-contact measurement of optical signals from the body. Due to the lack of coupling, even very subtle movement of an examined subject can result in very significant signal corruption. In terms of a Fourier-spectral analysis, a sharp signal form, being originated by motion artifact, would contribute over all the frequency ranges, and it is therefore very difficult to extract a biological signal by utilizing any frequency specific features, like as it is done for pulsatile signal, for example.

### SUMMARY OF THE INVENTION

There is a need in the art to provide a system for use in monitoring of biological parameters of a subject. The system includes (i) an illumination unit including at least one light source of at least one pre-selected wavelength band, to be applied to a selected region in the subject; and (ii) a detection system configured for measuring reflections of said light at different angles and different spatial locations with respect to the illuminated region. The detection unit is configured and operable to detect spatially separated light components corresponding to the specular dependent component of the signal and the pulsatile-related diffused component of the signal coming from the subject in different directions respectively, thereby defining at least two independent channels of information, enabling identification of the reflected signal part dependent on motion effects. The system includes a control unit connectable to said illumination unit and to said detection system, said control unit being configured to analyze at least two independent channels of information indicative of the detected signals, to eliminate the signal part dependent on motion effects and determine one or more biological parameters such as heart rate.

In some embodiments, the control unit includes:
a data acquisition utility responsive to data coming from said detection system; and
a modulating utility associated with the illumination unit;
a data processing and analyzing utility for analyzing data from said data acquisition utility and determining said at least one parameter;
a memory utility for storing coefficients required to perform predetermined calculation by said data processing and analyzing utility; and,
an external information exchange utility configured to enable downloading of the processed information to an external user.

The detection system may include at least one detection unit distant from one another detection units.

In some embodiments, the illumination unit is distantly located from the subject, and at least one of the detection units is attached to said subject. Alternatively, at least one of the detection and illumination units is distantly located from the subject. According to another embodiment, at least one of the detection units is distantly located from the subject.

The system may configured for use in sleep monitoring, and/or for use in Sudden Infant Death Syndrome monitoring and/or for use in patient monitoring at hospital condition, and/or for use in monitoring during sport activity.

The illumination unit may include at least one optically collimated light source, and a facility to direct the collimated beam to the selected region in the subject. The illumination unit is adapted to disperse the electromagnetic radiation so that part of it is scattered from the subject.

At least one source of the illumination unit may be coupled with a polarization unit enabling to create polarized electromagnetic signal in one preferable direction, and an entrance of at least one of detection units of the detection system is coupled with a polarization unit enabling only certain direction of pre-selected polarized radiation to be detected.

In some embodiments, the control unit is configured to analyze the data indicative of the detected signals and determine at least one blood related parameter of the subject, derive therefrom the at least one Central Nervous System (CNS) related characteristic, and compare said at least one CNS characteristic of the subject obtained prior to and under a provocation stimulus including exposure of the subject to pre-defined visual or audio information, which is chosen to be verified and revealed.

The system is configured and operable for distant or non-contact monitoring.

There is another broad aspect of the present invention to provide a method for use in non-invasive determination of biological parameters of a subject. The method includes illuminating a selected region of the subject by light of at least one wavelength, and detecting reflections of said light from at least two distant geometrical locations in said selected region, such as to detect spatially separated light components coming from the illuminated region in different directions respectively, thereby defining at least two independent channels of information, enabling identification of the reflected signal part dependent on motion effects.

The method may include distant or non-contact monitoring of a physiological parameter of a subject; exposing said subject to predefined stimulus; deriving central nervous system (CNS) characteristics from blood measurement; and comparing said CNS characteristics with CNS characteristics obtained prior the stimulus

Another aspect of the present invention is a method for extraction of biological signal out of noise and motion artifacts. The method includes using opto-physiological invariants (OPI) to distinguish between a real biological signal and other interferences. The method includes (i) building a set of the original signal being modified by different frequency sensitive band-pass filters; (ii) calculating said OPI for each band-pass ranges; and, (iii) extracting from the OPI data the frequency pattern of physiological signal value. The opto-physiological invariant may be GAMMA, defined as a ratio of (AC/DC)_{wavelength1}/(AC/DC)_{wavelength2} wherein (AC/DC) is the ratio of the pulsatile component of a signal to the mean value of the signal obtained for two different wavelengths, respectively. The OPI may also be a parametric slope (PS) associated with occlusion related signals, defined as (Δ Log(S₁)/Δ Log (S₂), where Δ Log(S₁) and Δ Log(S₁) are logarithmic time variations of light response signals S₁ and S₂ measured for two different wavelengths, respectively.

Alternatively, the OPI may be a linear or non-linear combination of GAMMA and PS for different combination of wavelengths. The OPI is a convolution of signal responses at different wavelengths.

One aspect of the present invention is associated with the fact that there are many medical conditions where a direct intermediate contact between a sensor and a subject's body is not advised or even impossible. The following are a few examples of such conditions:

The damage to epidermis and dermal elements from a burn injury creates a situation where any outside contact with a subject's body is associated with a risk of infection.

Application of optical measurements may produce skin damage after the administration of photosensitizing chemotherapeutic drugs.

Due to the course of complicated surgery, delivery, combat and terror causalities, a lot of sensors and vital sign monitors are attached to a subject body. Under such circumstances any available space around or nearby a subject becomes very important. All kinds of wires and inter-connections between the subject and outside devices can make difficult an essential free access of medical personal to the subject's body.

Under conditions of impaired immunities of the body, even small contamination of sensors can result in unpredictable infections. Examples of such a disease include: lupus rheumatoid, psoriasis, HIV, tuberculosis, eczema, viral and bacterial infections.

During prolonged monitoring of a sleep status under home or even laboratory environment, any contact between the sensor and subject has to be minimized. In this case, a distant monitoring will be helpful to secure a good sleep quality, on the one hand, and to provide a continuous monitoring of heart rate, oxygen saturation and other parameters essential as diagnostic and follow-up tools.

It is clear that under all these circumstances a medical system needs to be facilitated with means, enabling a distant or non-contact monitoring of a subject.

There are additional fields of application being characterized by strong motional artifacts. For example, Sudden Infant Death Syndrome (SIDS) is a medical condition in which an infant can stop breathing, which effect if being unobserved in time can lead to the death of the infant. However, attempts to address this problem by adaptation a conventional pulse oximeter (transmission or reflection mode) was found unpractical because of unacceptable level of false alarms, associated with uncontrollable baby's movement. A system that can measure pulse-related biological signal notwithstanding the baby's strong motion artifacts can be adopted for SIDS monitoring.

There is yet another, entirely different and non-medical field of application, where an ability to conduct a distant monitoring of a subject can be crucial. So-called lie detector or polygraph instrument is basically a combination of medical devices that are used to monitor changes occurring in the body. The variations of well-known medical parameters such as heart rate, respiratory rate, heart rate variability and others are implicated as a manifestation of reaction of a central nervous system (CNS). Fluctuations of the measured parameters may indicate that person is being deceptive. However, this test is rarely applied and its application is very restricted because of many practical and legal reasons. For example, thousands of people being passing the terminals prior to boarding their flights are obliged to pass the procedure of security control. All passengers are requested to answer a number of security-driven questions. However, it is not always possible to proceed with in-depth inquiry even for some suspicious subjects. In these cases the officials in charge have to make very subjective decisions whether the suspicious subject tells the truth or not. At this case, it would be very beneficial to be assisted by some real-time information indicating a degree of truthfulness of the answers the attendee is replied of. The best-case scenario is if the subject under examination is not aware of a fact that he is being tested. Such an examination can be performed only if a measurement of biological manifestations of CNS-functioning is done distantly and invisibly. Afterwards, this information can be processed and transferred to decision-makers.

Thus, the present invention provides for deriving the CNS characteristics from blood measurement. The latter is obtained for a subject as a base line and the CNS characteristics are measured for the subject while exposed to pre-defined visual or audio information, which is chosen to be verified and revealed and then compared to the CNS reactions prior provocation stimulus and after it is performed to reveal if said subject is aware of this pre-selected information. For example, an unrevealed, distant monitoring of HRV (heart rate variability) of a subject is carried out for a few minutes, in order to create a base line of HRV. At the next stage, the examined subject is exposed, without any previous notice, to a pre-prepared audio message, containing some kind of information, which may be recognized by the examined subject only if he is aware of this information. In case that the information (name of a certain person, for example) is recognized by this subject, the CNS sympathetic system will cause an immediate change of the HRV pattern, which will be detected by a surveillance system. This will help to find out whether an examined subject is aware of information which he is not supposed to be aware of. In this test, the different interference factors of standard “lie detector” tests where the subject is prepared to the test are overcome. It should be noted that the reaction of aware tested subject can lead to cognitive irregular CNS reaction, which can lead to misinterpretation of the test results. This problem is avoided by doing a distant test.

Considering contactless optical measurements, the underground physical assumptions are that light, scattered from perfused media, already contains the information about the blood related or specifically, the pulsatile component of the optical signal. In principle, the pulsatile signal can be used, as it is done in the classic photo-plethysmography measurement technique for oxygen saturation assessment. (The measurement has to be done by using illumination with at least two different wavelengths). Unfortunately, a real biological parameter, like arterial blood pulsation, is very difficult to extract while motion artifacts and noise corrupt the measured signal.

The inventor has found that optical radiation regarding in depth or bulk-related processes of blood perfusion and pulsation, after imposing strong motion artifacts, is transformed differently with respect to geometrical direction as compared to that of a non-bulk related part of the optical signal. The present invention takes advantage of this observation.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1-3 are schematic diagrams of different configurations of distant measurement systems;

FIGS. 4a-4b and 5a-5b graphically show an example of measurement of reflection signals using the system of FIG. 3;

FIG. 6 graphically shows the product of two Fourier spectrums being detected by Detection unit 1 and Detection unit 2;

FIG. 7 graphically shows time variations of two pulsatile signals S₁(t) and S₂(t) at two wavelengths respectively;

FIG. 8 represents GAMMAs values calculated from fragments of the signals of FIG. 7;

FIG. 9 shows the original pulsatile signal of FIG. 7 associated with noise and motion artifacts;

FIG. 10 shows the Fourier spectrum of the signal of FIG. 9;

FIGS. 11-24 show the histograms of GAMMAs values calculated for different band-pass ranges; and

FIG. 25 shows the peak of the GAMMAs value over all the frequency range.

### DETAILED DESCRIPTION OF THE PREFERRED EMBODIMENTS

The configuration and operation of the measurement system and the method of monitoring used therein can be better understood with reference to the drawings, wherein like reference numerals denote like elements through the several views and the accompanying description of non-limiting, exemplary embodiments.

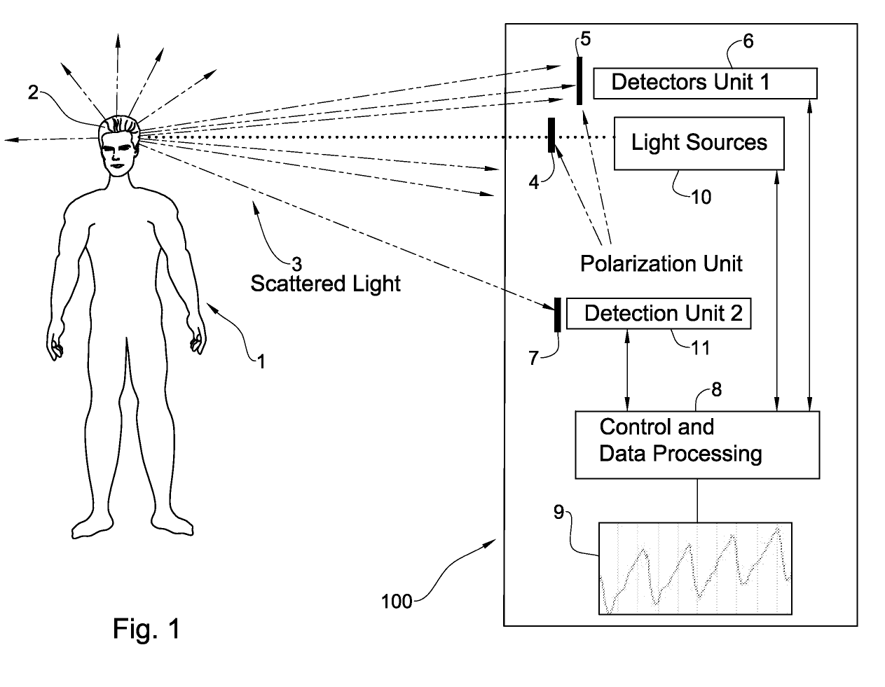

Reference is made to FIGS. 1 to 3 being schematic diagrams of different configurations of distant measurement systems. To facilitate understanding, the same reference numbers are used for identifying components that are common in all the examples.

All of these configurations include an illumination unit including at least one light source unit 10, and a detection system, which in the present examples includes two detection units 6 and 11. Light source unit 10 may include a multi-LED element, or a laser-diodes' array, or tunable laser, or a white light source with band-pass filters with shutters, or any combination of these light sources, enabling to illuminate a selected region of interest 2 (selected body part 2 of a subject 1) by using at least one wavelength.

It should be noted that the biological parameters of the subject may be selected from heart rate, arterial blood oxygen saturation, and other blood related parameters such as concentration of a substance in blood, blood flow, etc.

In some embodiments, the selected region of interest is illuminated with multiple wavelengths, for example selected for enabling determination of more than one biological parameter of the subject.

The measurement system 100 is associated with a control unit 8 that is configured to operate the light source unit 10. The control unit 8 is typically a computer system including inter alia a data acquisition utility responsive to data coming from said detection system; a modulating utility associated with the illumination unit; a data processing and analyzing utility for analyzing data from said data acquisition utility and determine said at least one parameter; and a memory utility for storing coefficients required to perform predetermined calculation by the data processing and analyzing utility; and preferably also an external information exchange utility configured to enable downloading of the processed information to an external user.

FIG. 1 shows an example of the system configuration, when the first detection unit 6 (Detection unit 1) and the second detection unit 11 (Detection unit 2) are oriented to collect light propagating from the illuminated region at different angles, respectively. In this example, detection unit 6 is located adjacent to the light source unit 10 (the axis of light collection by this detection unit forms a relatively small angle with the axis of propagation of the incident beam) and detection unit 11 is more distanced from the light source unit such that the axis of light collection by this detection unit 11 forms a relatively large angle with the incident beam propagation axis. Both detection units 6 and 11 are distant from the measurement location (from the region of interest).

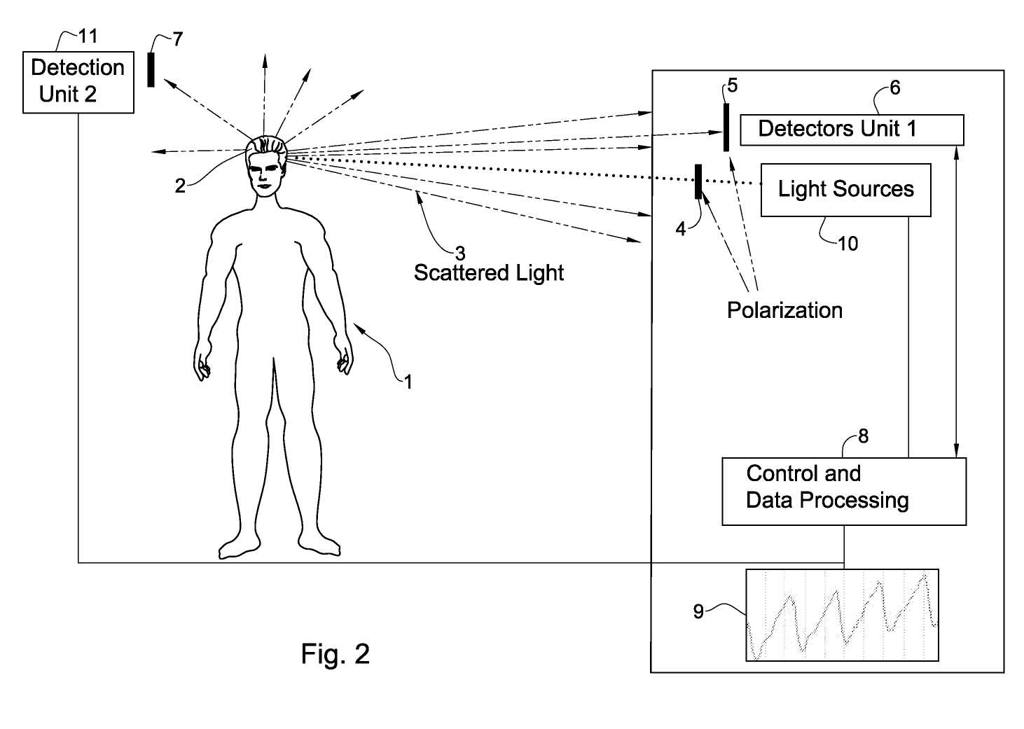

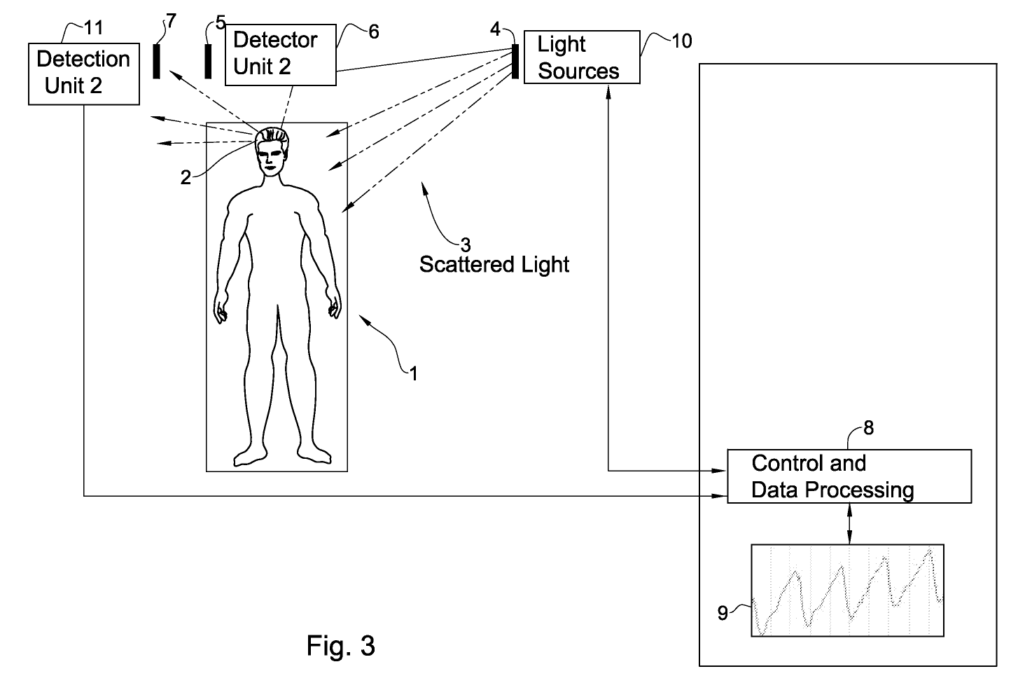

FIG. 2 shows another system configuration where one of the two detection units, Detection Unit 2, is located at close vicinity to the subject 1. FIG. 3 shows yet another configuration where both detection units are located at nearby space of the examined subject 1.

In all the examples, the different angles of collection by different detection units are such as to collect by one detection unit light specularly reflected from the illuminated region and collect by the other detection unit light scattered (diffused) by the illuminated region.

As shown in the example of FIG. 1, the optical radiation can be collimated on any part of the body, like the forehead 2 of the examined subject 1. In this case, an operator can be equipped with a camera and appropriately conjoined collimation system, and/or automatic image processing system, and operates to focus the collimated beam onto the selected region 2 in the subject 1.

In the system configuration of FIG. 3, the light source unit 10 is located in relatively close vicinity to the surrounding space where subject 1 is supposed to be located. Under this configuration, the light source unit 10 is configured to create a wide beam of radiation. The main advantage of this embodiment is that at least part of radiation falls on the skin of the examined subject 1, and thus the need for assistance of an image system or an operator is eliminated.

The system 100 may includes more than one light source unit, each of them being located at different points at subject surrounding space. This configuration is basically equivalent to a multi-detection system configuration, as will be described more specifically further below.

The distant measurement system includes at least two separate light detector units 6, 11 being significantly separated in a space, such as to detect spatially separated light components of light coming from the illuminated region of interest in different directions respectively. The detector unit includes a single detector or an array of detectors or CCD.

The geometric separation of the detection units to separately collect specular reflection and diffusion light components enables the differentiation and the elimination of the motion artifacts unavoidable in remote or distant measurement system.

It should be noted that the system of the present invention can also be used in a system/subject contact configuration, to minimize the motion artifact.

In some embodiments, at least two detection units are used. The detection units are spatially and angularly dissimilar to each other as much as possible.

It should be noted that plethysmography information comes from the depth of the skin and can be defined as so-called diffused component of a signal. The other part of a reflected signal is contributed by a direct specular reflection of light. The specular component of a signal contains less information about a pulse and is very sensitive to different motion artifacts, and therefore has to be eliminated.

It should be noted that the reflection of a specular component is governed by the Fresnels law. According to the Fresnel law, the variation of the reflected beam intensity is a function of the angle of incidence. On the other hand, the diffused component is not governed by Fresnel law but rather by the diffuse and transport equations for light propagating via blood and tissue. The manifestation of the some motion artifact by specular and diffused components is thus different in terms of time constants and signal amplitudes. Therefore, being measured at different angles and different spatial locations, the specular component of a signal behaves differently for each detector, whereas the pulsatile-related diffused component of a signal manifests very similar characteristics for all orientations and spatial locations.

The effect of difference between specular component and diffused components may be enhanced by using a polarization effect which is also strongly dependent on the geometry of reflected light detection. It should be noted that when light strikes a surface, the components of the electromagnetic field perpendicular and parallel to the plane of incidence get attenuated by different amounts. The degree of polarization of the reflected beam is a strong function of the angle of observation. Polarization means enables to differentiate between the two components of light, which behave differently at different angles. In some embodiments, the system includes light polarization add-ons.

Spatially separated detector units enable defining at least two independent channels of information. One component of the reflected signal is the pulsatile signal, originated by a subject. This component is geometrically invariant, whereas the specular-related component is highly dependent on motion effects. This multi-channel signal processing approach enables to discriminate noise and to enhance the biological signal of the body.

Typically, the specular-related component has the same polarization as the incident light. On the contrary, diffused reflected light component is depolarized. Therefore, it is possible to separate diffused components of the detected light out of the specular component. To be polarized the emitted light may pass through a liquid crystal unit or electro-optical phase modulator 4, as illustrated in FIGS. 1-3.

As illustrated in FIGS. 1-3, an incident polarized light beam illuminates the surface within the region of interest 2 (and is polarized according to one direction), and the reflected beams are simultaneously measured by the detection unit 6 and 11 at the orthogonal direction, by using appropriate polarization units 5 and 7 respectively. Using time varying polarization technique the ambient light radiation noises is strongly discriminated.

In some embodiments, a simple linear polarizer can be used to reduce the specular component of a signal. The diffused component related to a pulsatile signal 9 survives and is easily extracted.

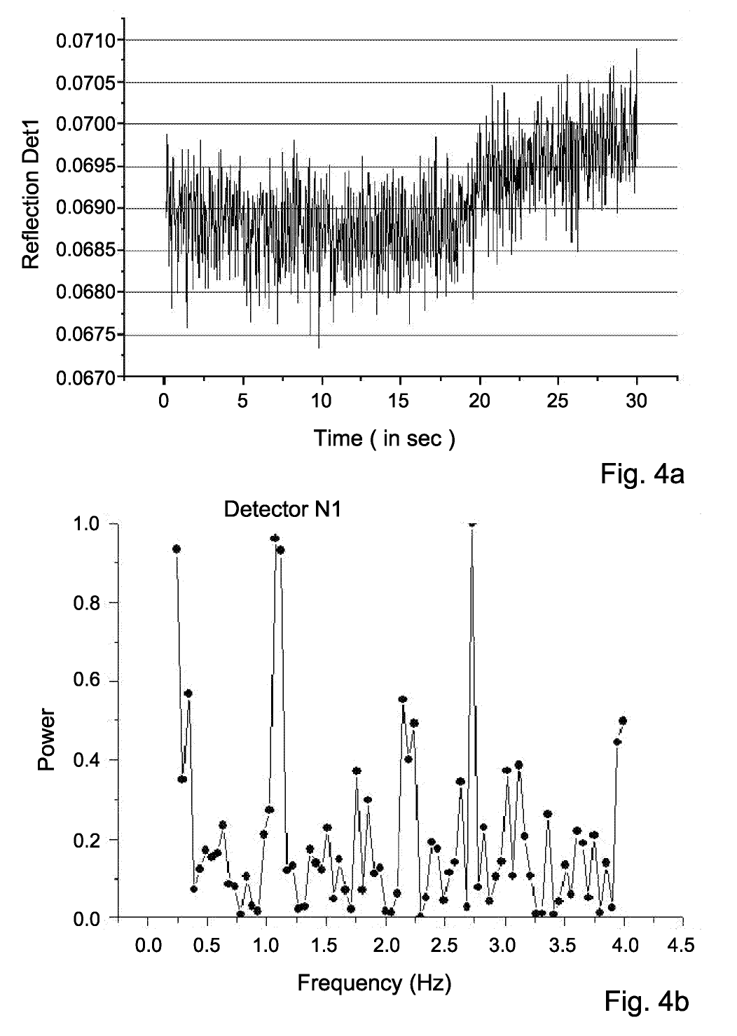

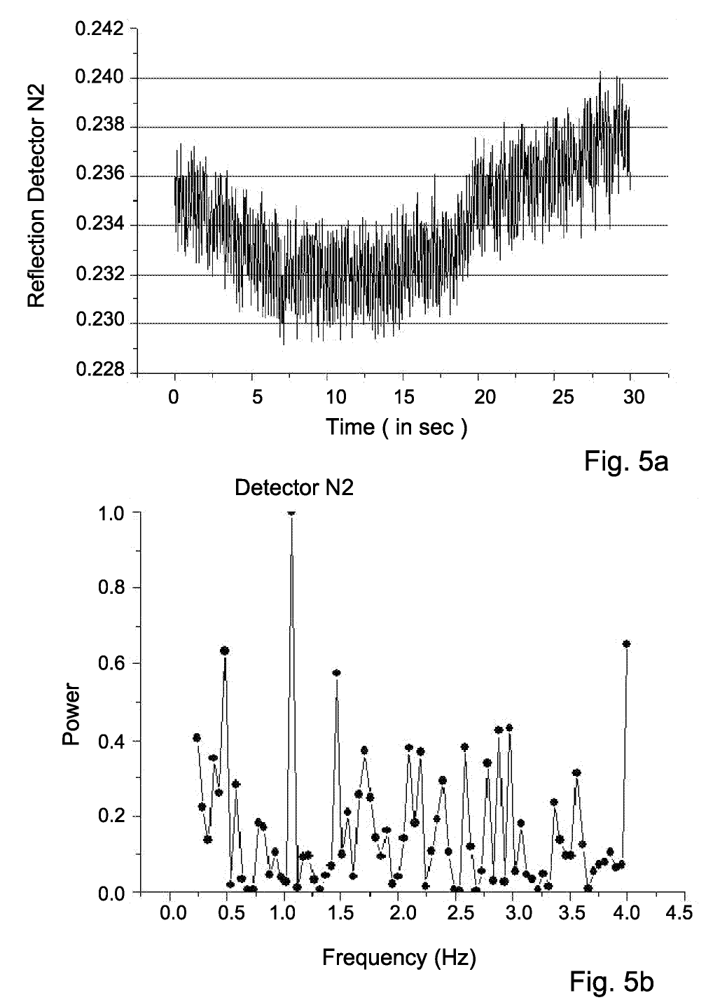

Reference is made to FIGS. 4a-4b and 5a-5b showing an example of measurement of reflection signals using the system shown in FIG. 3 while the reflection from subject's forehead 2 is measured by two detection modules 6 and 11. A drive unit (not shown) operates the LED-based light source unit to generate light of e.g. 810 nm, and reflection signals are collected remotely by using two separate detectors modules 6 and 11. The detected signal is digitized and stored for the next stage of analysis.

FIGS. 4a and 4b show, respectively, the time variation of the measured signal and a window of Fourier transform power spectrum of said signal, being detected by Detection unit 1. FIGS. 5a and 5b show similar results for the Detection unit 2.

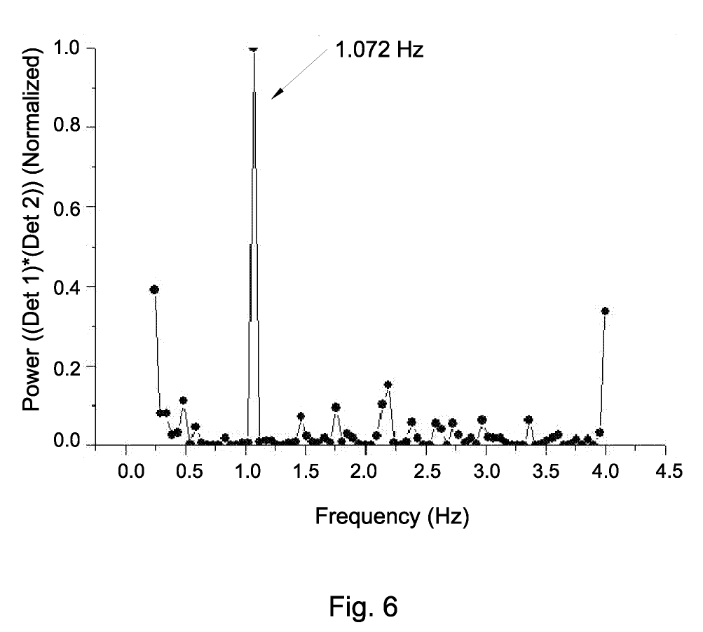

Reference is made to FIG. 6 showing the product of two Fourier spectrums giving a very prominent and sharp peak at 1.07 Hz, corresponding to 65 heart beats per minute. This result is confirmed by a reference standard pulse oximetry device.

The multi-detection technique can also be applied for non-distant measurement whereas the measurement system is entirely or partially attached to different regions on a subject. For example, in particular case of a baby's monitor, one sensor (illumination and detection units) can be attached to the finger, whereas another sensor can be attached to the forehead or to any other site of the body, such that the motion artifacts result in different kinds of signal perturbation at each locations. In this case, the convolution of spectrum for two detectors will cancel out motion artifacts because of different nature of artifacts at different body location, whereas the pulsatile signal is very similar for both sites.

There is another broad aspect of the invention to provide an optical spectrum-related method allowing for extracting the heart rate out of motion artifact and noise by using only one detector and a light source unit emitting more than one wavelength. This method takes advantage of the so-called opto-physiological invariants (OPI). The latter is defined here as any kind of mathematical transformation of measured optical responses so that the result of this mathematical transformation is almost independent on geometrical parameters of the measurement, but dependent only on physiological or biochemical properties of measured media or physiological process.

One example of such invariant is the so called parameter GAMMA which is defined as a ratio of (AC₁/DC₁)/(AC₂/DC₂), where AC₁/DC₁ is a ratio of pulsatile component (AC₁) of a signal to mean value of a signal (DC₁) obtained for wavelength λ₁ (for example 670 nm) and AC₂/DC₂ being a similar ration obtained for wavelength λ2 (940 nm, for example). GAMMA is independent upon any specific properties of a local site (finger size or skin properties) or upon measurement geometry. The only variable parameter, which corresponds to GAMMA, is arterial blood oxygen saturation (SPO2). Therefore, this parameter meets the criteria of OPI definition.

Another invariant of this kind is the so-called Parametric Slope (PS) and is associated with occlusion related signal. PS is derived from optical responses which are measured at two different wavelengths and is associated with SPO2, like GAMMA and can be defined as OPI. For example, PS is defined as Δ Log(S₁)/(Δ LogS₂), where Δ Log(S₁) and Δ Log(S₁) are logarithmic time variations of light signals S₁ and S₂ measured for two different wavelengths, respectively.

Linear or non-linear combination of GAMMA's and Parametric Slopes for more than two wavelengths, with pre-defined coefficients, can be defined as OPI, being associated with blood Hb. It is important to understand that a range of any specific OPI value is well defined by being a representation of an appropriate biological parameter.

Typically, the very basic principle of regular pulse-oximeter operation consists of measuring GAMMA from optical transmission or reflection signal and transforming the GAMMA value into SPO2 values, according to a predetermined calibration curve. In order to calculate the GAMMA value, at least two different wavelengths are used. The normal range of GAMMA value is restricted by a normal or physiological range of SPO2 values. For the combination of wavelengths 670 nm, 810 nm, a normal range is represented by GAMMA being between 0.55-0.6. Under acute situations, the GAMMA value can reach 0.8. Therefore, for healthy subject the GAMMA value can be fluctuated around 0.6. According to this method, the signal processing is initiated by calculation of GAMMA's or other OPI related functions.

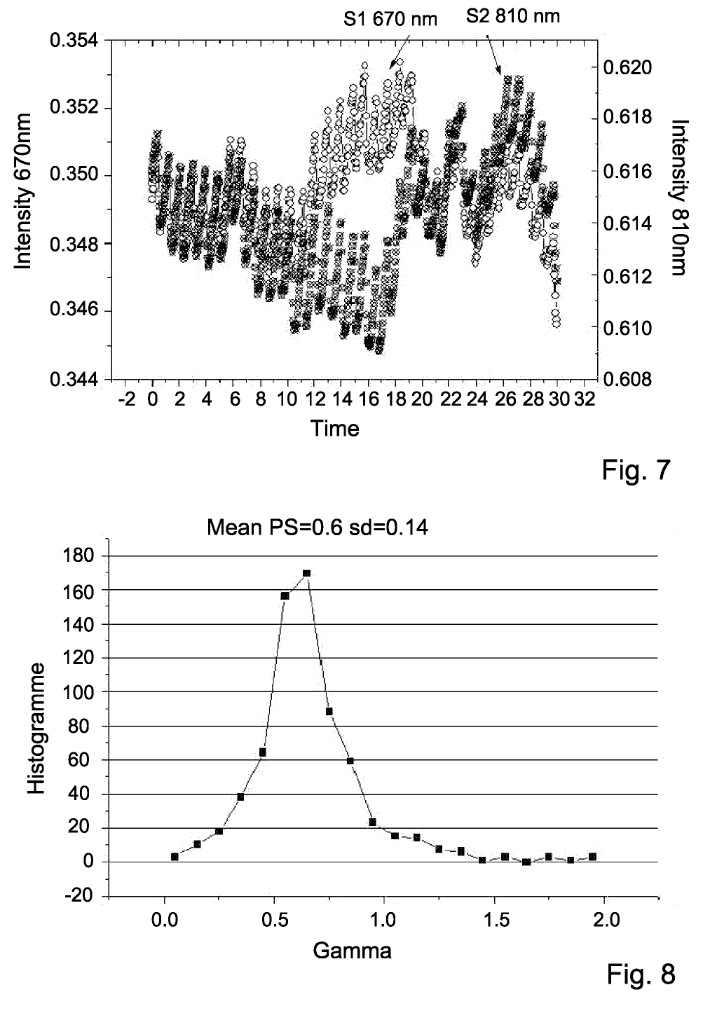

Reference is made to FIG. 7 showing time variations of two pulsatile signals S₁(t) and S₂(t) at two wavelengths, respectively, being measured concurrently from the forehead at rest position, without inducing motion artifacts and other noise (“original” signals). The heart rate frequency is about 1.1-1.2 Hz.

Reference is made to FIG. 8 showing a histogram representing GAMMAs values calculated from fragments of the signals of FIG. 7. The most probable value of GAMMA is 0.65, which corresponds to SPO2=96%, which is a physiologically acceptable value.

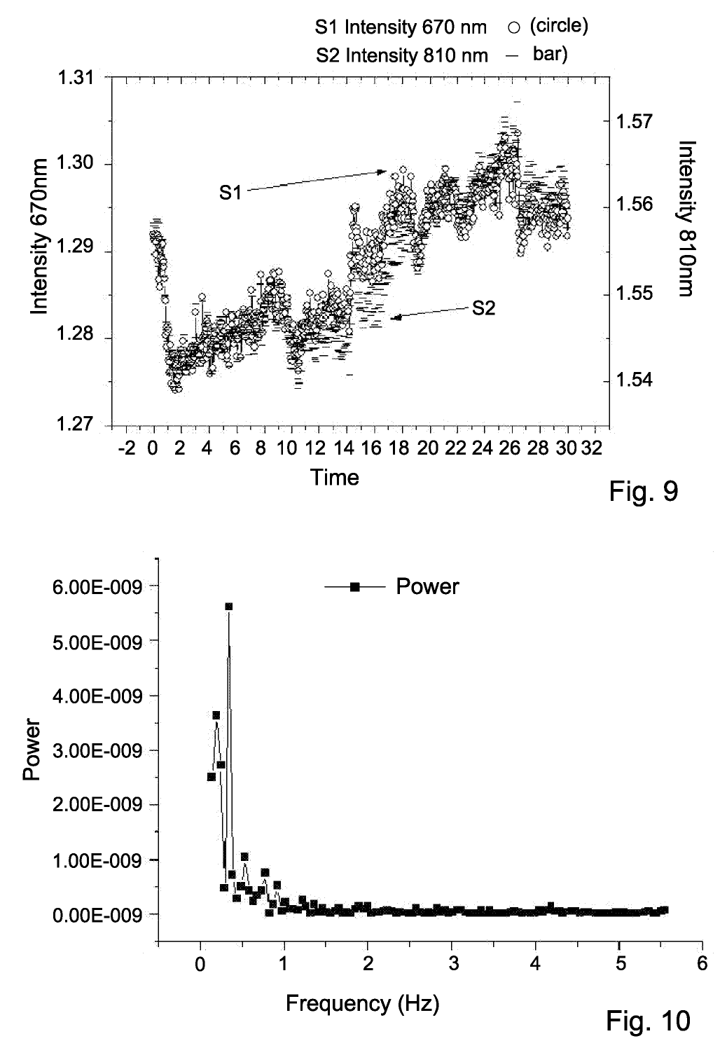

FIG. 9 shows how the original pulsatile signal (FIG. 7) is drastically corrupted by introducing some noise and motion artifacts.

FIG. 10 shows the Fourier spectrum of the signal of FIG. 9. The curve has no any prominent peak around 1.1-1.2 Hz as in the example of FIG. 7, and the commonly used signal processing techniques is not useful to derive the real heart rate. However, the technique of the present invention using OPI enables to easily extract this information. The first step is in building a set of the original signal being modified by different frequency sensitive band-pass filters. At this example, a set of digital FFT based band-pass windows with width of 0.1 Hz ranging from 0.5 Hz up to 2 Hz was used. The signal was passed alternatively through each of these band-passes.

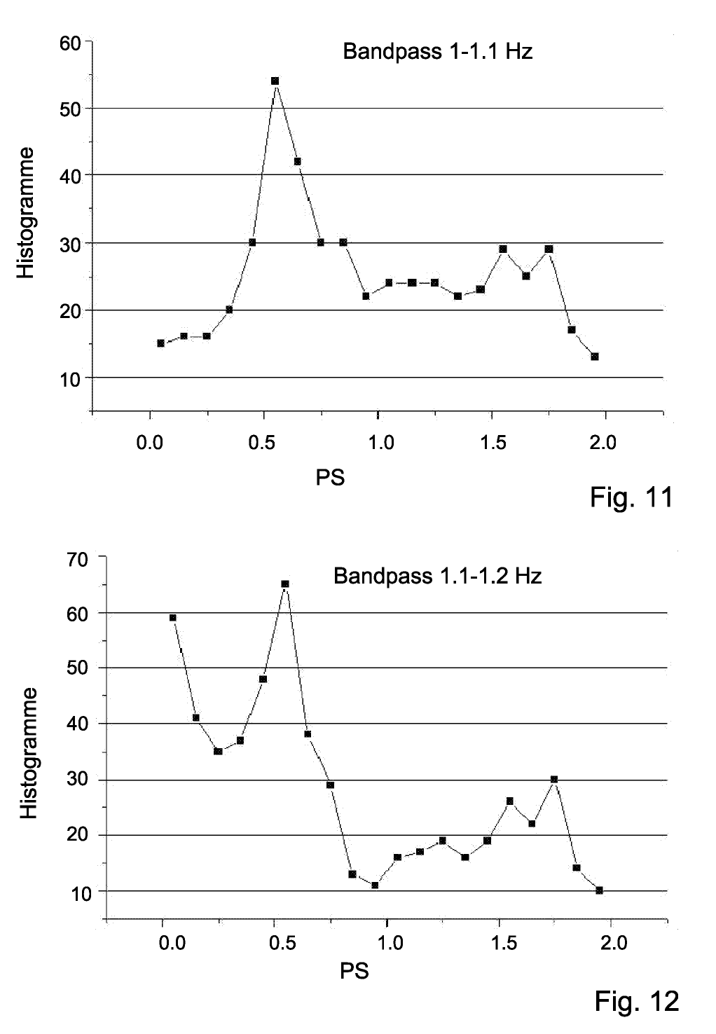

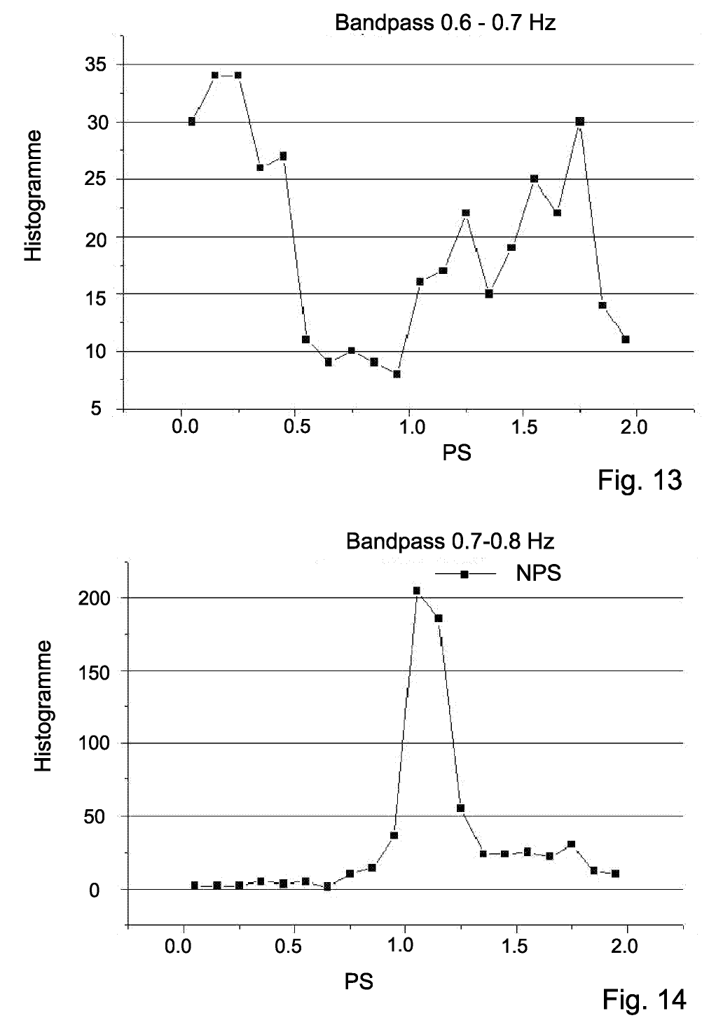

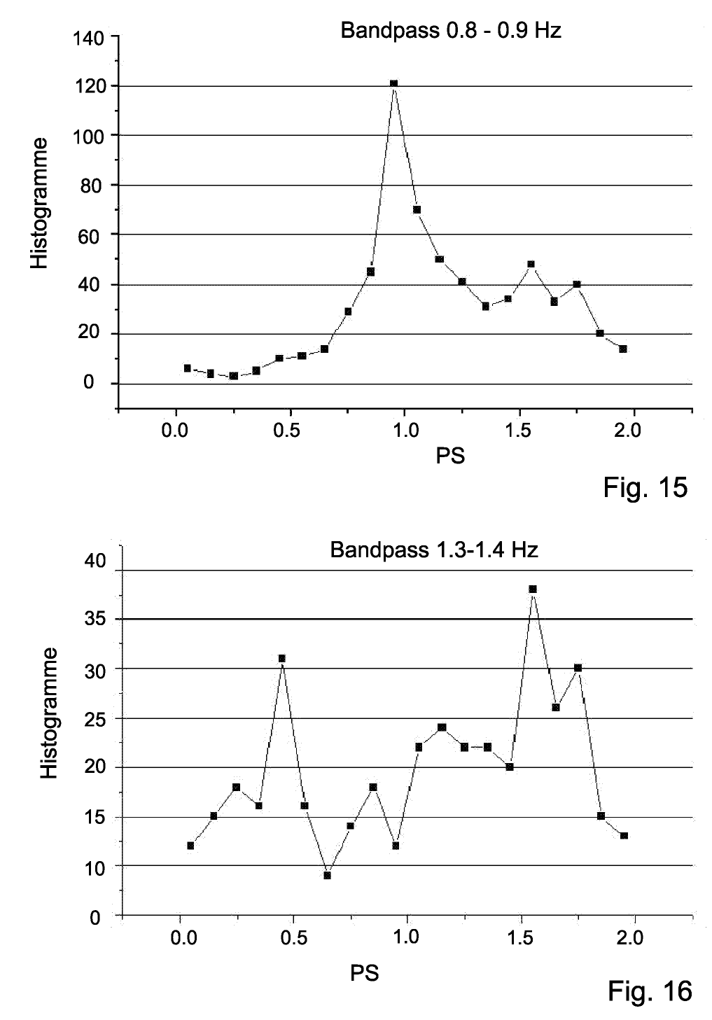

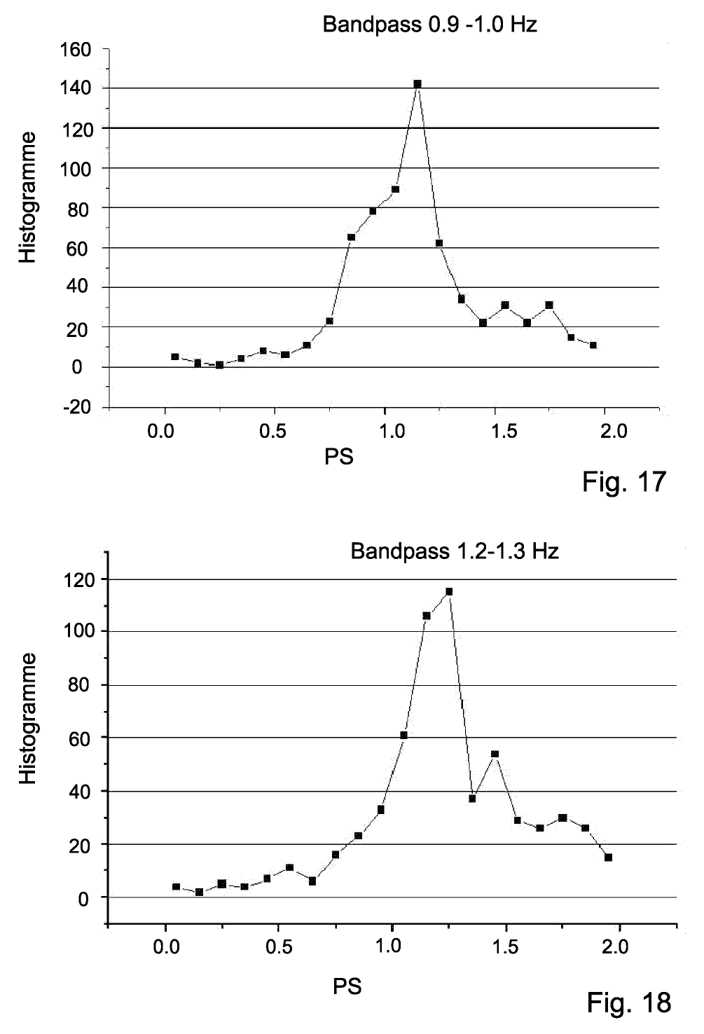

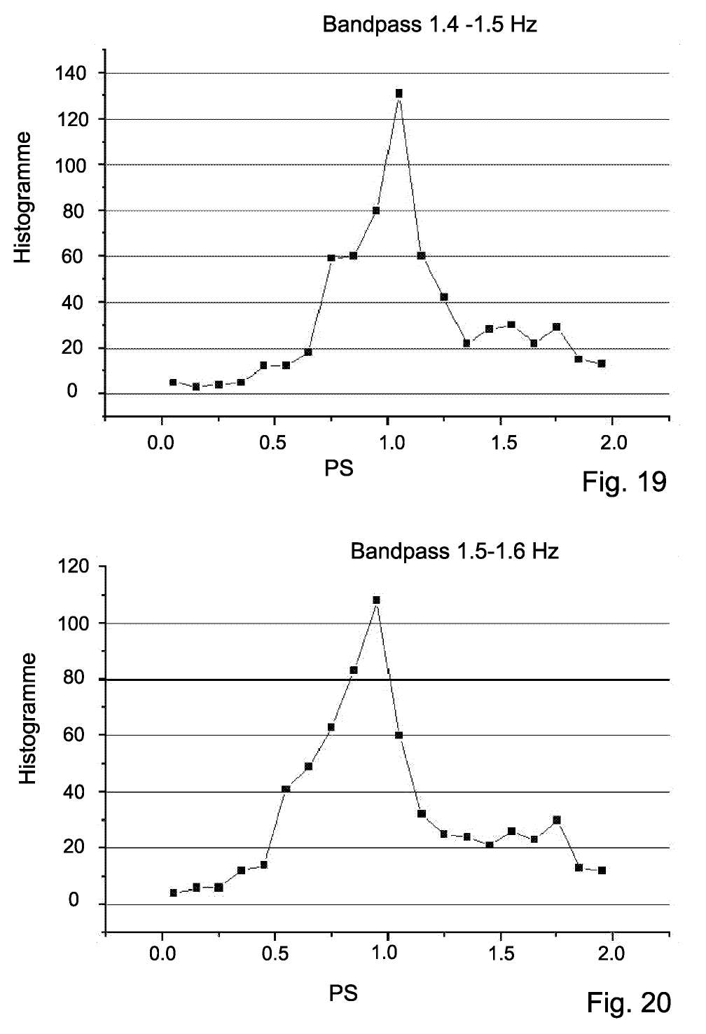

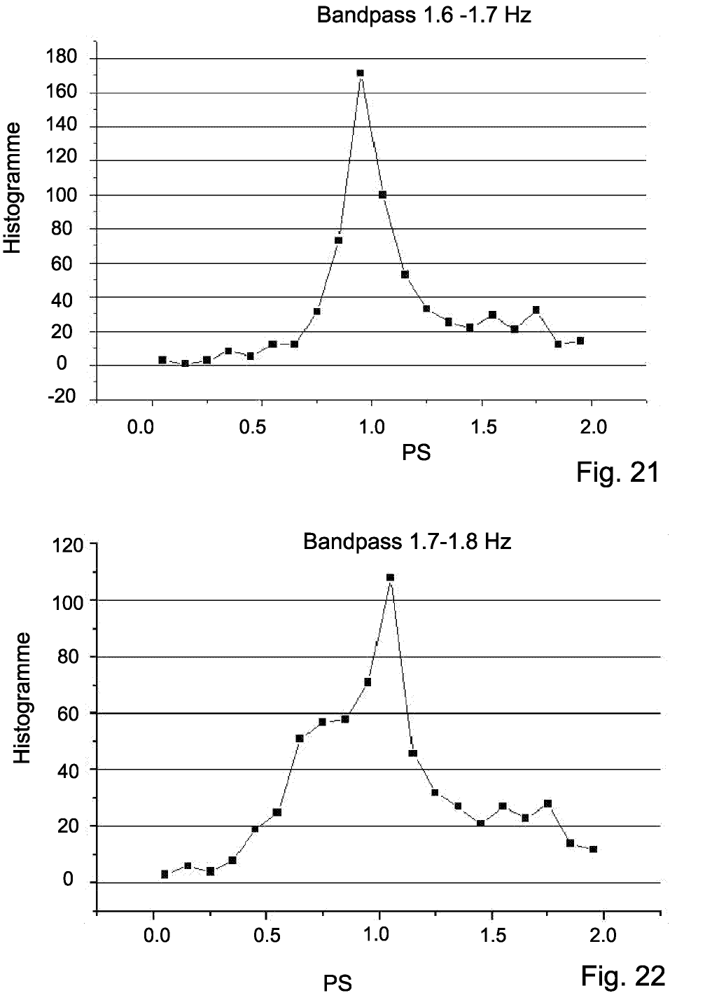

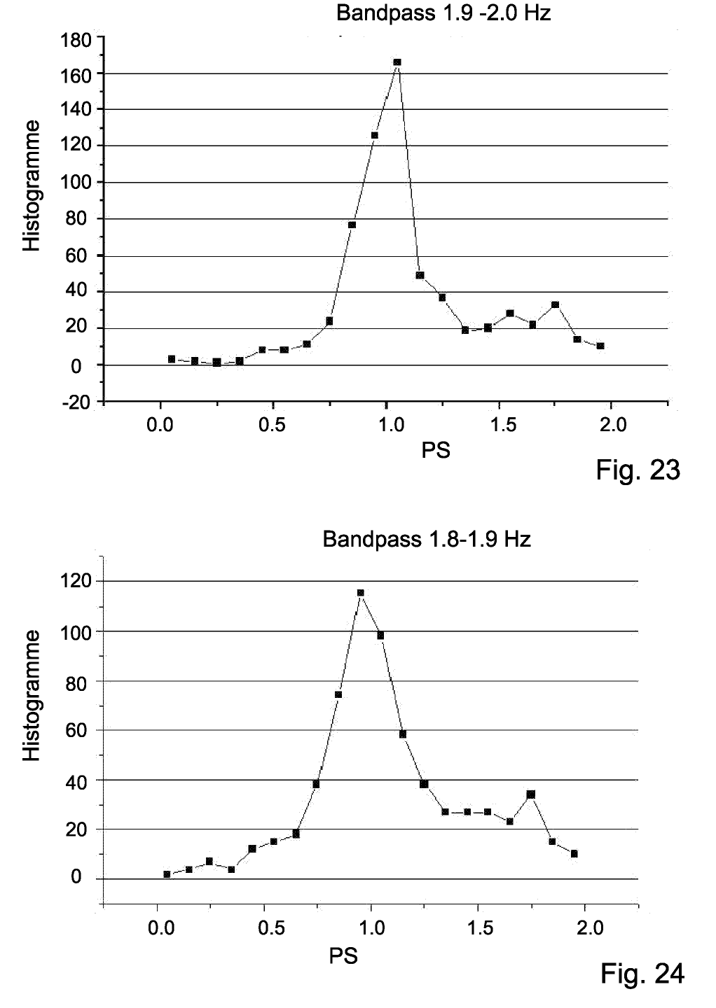

FIGS. 11-24 show the histograms of GAMMA's, as calculated for band-pass signals for different band-pass ranges.

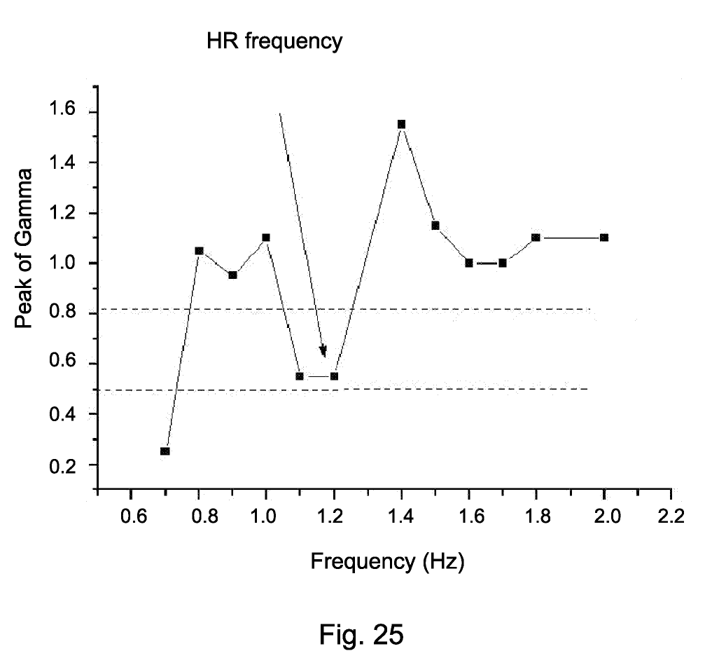

FIG. 25 shows peak of GAMMAs as a function of frequency. As explained above, the normal range of GAMMA value is restricted by a normal or physiological range of SPO2 values. For the combination of wavelengths 670 nm, 810 nm, a normal range of GAMMA value is about 0.55-0.8. The only peak of GAMMA which matches with this physiological range is located between 1.1-1.2 Hz. The signal frequencies associated with the GAMMA's values beyond this physiological range are related to noise or motion artifacts. In this particular example, the range 1.1-1.2 Hz corresponds to a heart rate interval of 66-72 beat's per minute. This interval corresponds to the interval of the heart rate measured independently. Therefore, the technique of the present invention enables to distinguish between the actual heart beats rate and any kind of unrelated noise.

It should be noted that the technique of the present invention can be applied for different OPI. To increase the accuracy and the reliability of this technique; this method can be associated with the measurement system as described above.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system for use in monitoring of biological parameters of a subject, the system comprising:
(i) an illumination unit including at least one light source of at least one pre-selected wavelength band, to be applied to a selected region in the subject; and
(ii) a detection system configured for measuring reflections of said light at different angles and different spatial locations with respect to the illuminated region, said detection system being configured and operable to detect spatially separated light components corresponding to the specular dependent component of the signal and the pulsatile-related diffused component of the signal coming from the subject in different directions respectively, thereby defining at least two independent channels of information, enabling identification of the reflected signal part dependent on motion effects.

2. The system of claim 1, comprising a control unit connectable to said illumination unit and to said detection system, said control unit being configured to analyze at least two independent channels of information indicative of the detected signals, to eliminate the signal part dependent on motion effects and determine one or more biological parameters.

3. The system of claim 2, wherein the control unit comprises:
a data acquisition utility responsive to data coming from said detection system; and
a modulating utility associated with the illumination system;
a data processing and analyzing utility for analyzing data from said data acquisition utility and determining said at least one parameter;
a memory utility for storing coefficients required to perform predetermined calculation by said data processing and analyzing utility; and,
an external information exchange utility configured to enable downloading of the processed information to an external user.

4. The system of claim 1, wherein the detection system comprises at least one detection unit distant from one another detection units.

5. The system of claim 4, wherein the illumination unit is distantly located from said subject, and at least one of the detection units is attached to said subject.

6. The system of claim 1 wherein at least one of the detection and illumination units is distantly located from the subject.

7. The system of claim 4, wherein at least one of the detection units is distantly located from the subject.

8. The system of claim 1, configured for use in sleep monitoring.

9. The system of claim 1, configured for use in Sudden Infant Death Syndrome monitoring.

10. The system of claim 1, configured for use in patient monitoring at hospital condition.

11. The system of claim 1, configured for use in monitoring during sport activity.

12. The system of claim 1, wherein said illumination unit includes at least one optically collimated light source, and a facility to direct said collimated beam to said selected region in the subject.

13. The system of claim 1, wherein said illumination unit is adapted to disperse the electromagnetic radiation so that part of it is scattered from said subject.

14. The system of claim 1, wherein said at least one source of the illumination unit is coupled with a polarization system enabling to create polarized electromagnetic signal in one preferable direction, and an entrance of at least one of detection units of the detection system is coupled with a polarization system enabling only certain direction of pre-selected polarized radiation to be detected.

15. The system of claim 1, wherein said at least one parameter of the examined subject is heart rate.

16. The system of claim 2, wherein the control unit is configured to analyze the data indicative of the detected signals and determine at least one blood related parameter of the subject, derive therefrom the at least one Central Nervous System (CNS) related characteristic, and compare said at least one CNS characteristic of the subject obtained prior to and under a provocation stimulus including exposure of the subject to pre-defined visual or audio information, which is chosen to be verified and revealed.

17. The system of claim 1, configured and operable for distant or non-contact monitoring.

18. A method for use in non-invasive determination of biological parameters of a subject, the method comprising illuminating a selected region of the subject by light of at least one wavelength, and detecting reflections of said light from at least two distant geometrical locations in said selected region, such as to detect spatially separated light components coming from the illuminated region in different directions respectively, thereby defining at least two independent channels of information, enabling identification of the reflected signal part dependent on motion effects.

19. The method of claim 18, comprising distant or non-contact monitoring of a physiological parameter of a subject; exposing said subject to predefined stimulus; deriving central nervous system (CNS) characteristics from blood measurement; and comparing said CNS characteristics with CNS characteristics obtained prior the stimulus

20. A method for extraction of biological signal out of noise and motion artifacts, the method comprising using opto-physiological invariants (OPI) to distinguish between a real biological signal and other interferences.

21. The method of claim 20, comprising:
building a set of the original signal being modified by different frequency sensitive band-pass filters;
calculating said OPI for each band-pass ranges; and,
extracting from the OPI data the frequency pattern of physiological signal value.

22. The method of claim 20, wherein said opto-physiological invariant is GAMMA, defined as a ratio of (AC/DC)_{wavelength1}/(AC/DC)_{wavelength2} wherein (AC/DC) is the ratio of the pulsatile component of a signal to the mean value of the signal obtained for two different wavelengths, respectively.

23. The method of claim 20, wherein said OPI is a parametric slope (PS) associated with occlusion related signals, defined as (Δ Log(S₁)/Δ Log (S₂), where Δ Log(S₁) and Δ Log(S₁) are logarithmic time variations of light response signals S₁ and S₂ measured for two different wavelengths, respectively.

24. The method of claim 20, wherein said OPI is a linear or non-linear combination of GAMMA and PS for different combination of wavelengths.

25. The method of claim 20, wherein said OPI is a convolution of signal responses at different wavelengths.
